(19) United States
(12) Patent Application Publication
(10) Pub. No.: US 2025/0345336 A1
(43) Pub. Date: Nov. 13, 2025

(54) ALLOPURINOL ORAL SUSPENSION

(71) Applicant: Extrovis AG, Baar (CH)

(72) Inventors: Kirankumar Kowkuntla, Kendall Park, NJ (US); Rajasekhara Reddy Singampalli, Hyderabad (IN)

(21) Appl. No.: 19/203,383

(22) Filed: May 9, 2025

(30) Foreign Application Priority Data
May 10, 2024 (IN) 202441037212

(51) Int. Cl. A61K 31/519; A61K 9/00; A61K 47/10; A61K 47/12; A61K 47/14; A61K 47/26; A61K 47/34; A61K 47/36

(52) U.S. Cl. CPC A61K 31/519; A61K 9/0056; A61K 47/10; A61K 47/12; A61K 47/14; A61K 47/26; A61K 47/34; A61K 47/36

(57) ABSTRACT

The present disclosure provides an oral suspension of allopurinol suitable for administration to pediatric, geriatric patients, as well as to patients experiencing difficulty in swallowing solid dosage forms. The suspension is stable, palatable and safer compared to compounded suspensions of allopurinol. The suspension dosage form of allopurinol also allows for a more accurate dosing. The suspension is stable for at least 24 months at room temperature, demonstrating low impurity formation under storage conditions.

### FIELD OF THE INVENTION

The present disclosure relates to the field of pharmaceutical formulations, and more specifically to a stable and palatable oral suspension of allopurinol suitable for administration to pediatric, geriatric, and dysphagic patients.

### BACKGROUND OF THE INVENTION

Allopurinol, chemically known as 1, 5-dihydro-4H-pyrazolo [3, 4-d] pyrimidin-4-one, is a xanthine oxidase inhibitor, having structure of formula as follows:

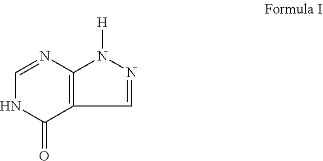

Allopurinol is a structural analogue of the natural purine base, hypoxanthine. It acts on purine catabolism, without disrupting the biosynthesis of purines. It reduces the production of uric acid by inhibiting the biochemical reactions immediately preceding its formation. It is an inhibitor of xanthine oxidase, the enzyme responsible for the conversion of hypoxanthine to xanthine and of xanthine to uric acid, the end product of purine metabolism in humans. Allopurinol is metabolized to the corresponding xanthine analogue, oxypurinol (alloxanthine), which also is an inhibitor of xanthine oxidase.

The USFDA has approved allopurinol tablets for (1) use in the management of adults with signs and symptoms of primary or secondary gout (acute attacks, tophi, joint destruction, uric acid lithiasis, and/or nephropathy), (2) the management of adult and pediatric patients with leukemia, lymphoma and solid tumor malignancies who are receiving cancer therapy which causes elevations of serum and urinary uric acid levels, (3) the management of adult patients with recurrent calcium oxalate calculi whose daily uric acid excretion exceeds 800 mg/day in male patients and 750 mg/day in female patients, despite lifestyle changes (such as reduction of dietary sodium, non-dairy animal protein, oxylate rich foods, refined sugars and increases in oral fluids and fruits and vegetables). An injectable formulation of allopurinol sodium is also approved, although for use in management of adult and pediatric patients with leukemia, lymphoma, and solid tumor malignancies who are receiving cancer therapy which causes elevations of serum and urinary uric acid levels and who cannot tolerate oral therapy.

There is no approved liquid dosage form of allopurinol on the market to date. As a result, administration of tablets to pediatric patients is a challenge, which is being solved by crushing allopurinol tablets and suspending them in vehicles suitable for oral administration.

Alexander et al, “Stability of Allopurinol Suspension Compounded from Tablets”, International Journal of Pharmaceutical Compounding Vol. 1 No. 2 March/April 1997, discloses a compounded suspension of allopurinol, prepared by crushing allopurinol tablets and incorporating the fine powder, in portions, with equal amounts of previously hydrated sodium carboxymethylcellulose and Veegum®. A syrup vehicle that is 75% w/w maltilol (Lycasin®) was added slowly to this mixture with continuous mixing until a homogeneous mixture was obtained. A 60-mL portion of paraben concentrate, which contained methylparaben 10% and propylparaben 2% in propylene glycol was slowly added to the mixture. Sodium bisulfite 1.5 g and sodium saccharin 1.5 g were dissolved in distilled water 75 mL; this solution was transferred to the mortar and the mixture was homogenized. Wild cherry flavoring 2.0 mL and vanilla flavoring 2.0 ml were added and mixed. The contents of the mortar were transferred to a graduated cylinder and distilled water was used to bring the volume to 3000 mL. While the reference states that the compounded allopurinol suspension was found to be stable and is commercially feasible, it is clear that the process of crushing tablets and then suspending it in a typical compounding pharmacy like environment has the risk of contamination and infections arising therefrom. Further, nothing in this reference provides any details on the particle size of allopurinol to be used to be able to achieve the desired efficacy; there is no discussion on the release of the allopurinol. Also, there is no detailing on caking or settling of the allopurinol over a long period of time. While the reference states that—“All samples remained homogeneous and showed no signs of caking or settling. Redispersion was accomplished by simply inverting and shaking the suspension bottle a few times.”—this conclusion has been derived on the basis of the 97-days study conducted. There is nothing in the reference that teaches that the product would have desirable properties at the end of a shelf life of 24 months or more.

Dressman et al, “Stability of Allopurinol and of Five Antineoplastics in Suspension”, Am J Hosp Pharm. 1983; 40:616-8, also discloses a suspension of allopurinol obtained by crushing tablets of allopurinol, and suspending the same in a vehicle with the help of a suspending agent (Cologel). The reference states that the suspension so obtained is stable for at least 56 days, i.e. the assay stays above 90% for at least 56 days. Nothing in this reference provides a confidence that the suspension will be table, free of contamination and that it can be safely used over a longer period, such as a shelf life of 24 months or more. Further, there is no details provided on the viscosity of the suspension, or the particle size of allopurinol, or the release of allopurinol from the suspension. In fact, the reference concludes with—“Because of caking problems, it was necessary to use ultrasound to aid recovery of drug from these samples. Therefore, the low assay values were thought to reflect a recovery problem rather than degradation of the drug. In these cases where refrigeration increased caking, it may be preferable to store the suspension at room temperature or design an alternative formulation.” Clearly then Dressman et al does not teach a shelf stable suspension formulation of allopurinol.

Polonini et al, “Stability of Allopurinol, Amitriptyline Hydrochloride, Carbamazepine, Domperidone, Isoniazid, Ketoconazole, Lisinopril, Naproxen, Paracetamol (Acetaminophen), and Sertraline Hydrochloride in SyrSpend SF PH4 Oral Suspensions” International Journal of Pharmaceutical Compounding, 1 Sep. 2016, 20(5):426-434, teaches the use of ready-to-use suspending vehicles such as SyrSpend SF PH4 as a suitable resource for suspending crushed tablets of allopurinol, to obtain a suspension suitable for administration to elderly and pediatric patients that present dysphagia. The beyond-use date for the suspension was found to be 90 days, i.e. the stability, and therefore usability, of the suspensions could not be guaranteed beyond 90 days.

Pramar et al,“Physicochemical Stability of Compounded Allopurinol Suspensions in PCCA Base, SuspendIt” Int J Pharm Compd. 2020; 24(5):413-419, also discloses allopurinol suspension obtained by crushing tablet of allopurinol and suspending the powder so obtained in SuspendIt at 10.0 mg/mL and 20.0 mg/mL concentrations. While this reference teaches that a suspension obtained as described here has a stability up to 180 days, i.e. a beyond-use date of 180 days, there is nothing in there to teach or suggest the formulation of a suspension that is stable and efficacious over a shelf life of 24 months or more. Nothing in this reference talks about the viscosity of the formulation, particle size of allopurinol to use and/or the release of allopurinol from the suspension.

Thus, while the prior art is replete with references of compounded suspensions of allopurinol, there is no commercially available product. There is a long-felt need for an allopurinol suspension that is safe for use in patients, and which has a long shelf life of 24 months or more. There is also a need for an allopurinol suspension that is not compounded at a pharmacy, and is safe because of lack of risk of contamination, and under or over dosage, which is a common error at compounding pharmacies.

### OBJECTIVE OF THE INVENTION

An object of the present disclosure is to provide a stable, ready-to-use and palatable oral suspension of allopurinol.

Another object of the present disclosure is to provide an oral suspension of allopurinol to improve dosing accuracy and ease of administration for patients with swallowing difficulties.

Yet another object of the present disclosure is to provide an oral suspension of allopurinol that is stable for at least 24 months, when stored at room temperature. These and other objects and advantages of the present disclosure will be apparent from the ensuing description.

### SUMMARY OF THE INVENTION

The present disclosure provides an an oral pharmaceutical suspension comprising about 10 mg/ml to about 20 mg/ml of allopurinol, and one or more pharmaceutically acceptable excipients.

In one embodiment, the oral suspension of allopurinol contains allopurinol having a particle size such that 90% of the allopurinol particles in the suspension have a particle size less than about 70 microns.

In one embodiment, the oral suspension of allopurinol has a viscosity ranging from about 80 mPa·s to about 130 mPa·s

In another embodiment, the oral suspension of allopurinol releases not less than 75% of total allopurinol within 15 minutes, when tested using USP Type II apparatus in 900 ml of 0.01N hydrochloric acid at 50 rpm.

In yet another embodiment, the oral suspension of allopurinol is such the sedimentation volume is not less than 0.9.

In another embodiment, the pH of the suspension is between about 3.0 to about 6.0 and viscosity is between about 80 mPa·s to about 130 mPa·s.

In one embodiment of the present disclosure, the pharmaceutically acceptable excipients are one or more excipients selected from a group comprising solvents, cosolvents, suspending agents, sweeteners, pH adjusting agents, flavoring agents, preservatives and anti-foaming agents.

The allopurinol suspension of the present disclosure is stable and does not degrade, i.e. it has acceptable levels of impurities when stored at room temperature for 24 months or more.

The allopurinol suspension is suitable for administration to pediatric and geriatric patients, as well as to patients that have difficulty in swallowing solid dosage forms, such as those that are bedridden, or have undergone surgery and/or are required to stay in bed.

### DETAILED DESCRIPTION OF THE INVENTION

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present disclosure also contemplates that in some embodiments of the invention, any feature or combination of features set forth herein can be excluded or omitted.

All publications, patent applications, patents, and other references mentioned herein are incorporated by reference in their entirety for all purposes.

As used herein, “a,” “an,” or “the” can mean one or more than one.

Furthermore, the term “about,” as used herein, when referring to a measurable value such as an amount of a compound or agent of this invention, dose, time, temperature, and the like, is meant to encompass variations of #10% of the specified amount.

The term “comprising”, which is synonymous with “including”, “containing”, or “characterized by” herein defined as being inclusive or open-ended and does not exclude additional, unrecited elements or method steps, unless the context clearly requires otherwise.

Also as used herein, “and/or” refers to and encompasses any and all possible combinations of one or more of the associated listed items, as well as the lack of combinations when interpreted in the alternative (“or”).

The term “excipient” as used herein refers to a component that can be added in the composition, which is physiologically tolerable and does not typically produce allergic or similar untoward reaction. The excipient also provides improved stability and therapeutic enhancement to the composition. The excipients may be added to the composition to facilitate manufacturing process, enhance stability, control release, enhance product characteristics, enhance bioavailability, enhance patient acceptability, or the like. The term “excipient” and “pharmaceutically acceptable excipient” shall be used interchangeably.

The United States Pharmacopoeia (USP) defines stability as “the ability of a product to retain its characteristics that it possessed during its manufacturing (physical, chemical, microbiological, therapeutic properties) within specified limits throughout its period of storage and use”. According to the ICH guidelines, pharmaceutical stability testing is defined as “systematic experiments conducted on pharmaceutical products to understand and provide evidence how the quality of a drug product varies under the influence of a variety of environmental factors such as temperature, humidity and light, and to set a re-test period for the drug, or a shelf life for the drug product and recommend good storage conditions”. In view of these, degradation products in a drug product are required to be evaluated and reported to the regulatory agencies. Further, USFDA requires formulations to be stable for a minimum period of 12 months (i.e. 48 weeks) at recommended storage condition, for approving the same for commercial use. For products to be commercially marketed in the US, there is an expectation that the products will comply with the standard throughout the shelf life.

Thus, the composition must have an acceptable level of impurity. The term “acceptable level of impurity” refers to the impurities in drug products not exceeding the permitted daily exposure (PDE), and/or impurities within limits defined by ICH for pharmaceutical products. “Stable”, as used herein, refers to physical and chemical stability of allopurinol in the aqueous solution, wherein the assay values remain within ±10% of the initial assay value, and the total impurities remain at less than 0.5% w/w of the composition, upon storage of the composition at 40° C./75% relative humidity (RH) for up to 6 months, and/or at 25° C./60% RH for up to 6 months.

The term ‘stable’ also encompasses a composition that has a shelf-life of at least 12 months. The term “shelf life” refers to the amount of time the pharmaceutical composition may be stored without loss of potency and/or performance profile. In some embodiments of the present disclosure, shelf life refers to the amount of time the composition may be stored with loss of not more than about 5%, preferably not more than about 4%, preferably note more than about 3%, preferably not more than about 2% or still preferably not more than about 1% of the potency and/or performance, when stored at room temperature of 25° C. and 60% relative humidity. The stable composition provided herein is designed to have a shelf life of at least about 12 months, preferably about 15 months, more preferably about 18 months, even more preferably about 24 months.

A pharmaceutical ‘suspension(s)’ is a heterogeneous system in which finely divided solid particles are dispersed in a liquid medium. Unlike solutions, where solutes are completely dissolved, suspensions involve particles that are only partially soluble or insoluble in the liquid. The term suspension(s), composition(s), product(s), preparation(s) and formulation(s) have been used interchangeably herein.

A ‘compounded suspension’ is a type of medication prepared by a pharmacist or compounding pharmacy by mixing ingredients, often including a drug powder, with a suspending vehicle like syrup or water. This process creates a liquid formulation where the drug particles are suspended within the liquid rather than being dissolved, like in a solution. It must be noted that compounded suspensions are produced on a small scale, suitable for use in a particular patient, and are suitable for use for upto 90 days, or at most 180 days from the day of compounding. This is typically known as “BUD” or best use date, which indicates that the compounded suspension is best for use by and before the date specified, which as mentioned above, is usually 90 to 180 days. The suspension has to be discarded after such a date, and use after such a date may actually have side effects, and/or lower therapeutic effects.

The term “caking” in the context of the present disclosure refers to hard, compact sediment of the drug in the suspension, at the bottom of the container, that is difficult or impossible to redisperse by normal shaking.

Allopurinol is a xanthine oxidase inhibitor commonly used for the treatment of gout, hyperuricemia, and uric acid nephropathy. Commercially available formulations of allopurinol are typically available in tablet form or injection form, which may not be suitable for pediatric, geriatric or dysphagic patients who require liquid dosage forms.

The only alternative for such patients is to use a suspension obtained by crushing tablets of allopurinol and suspending the powder in a suitable suspending vehicle. However, such a compounded suspension is subject to lack of stability, or stability over a shorter time frame. Further, a compounded product is subject to several issues such as possibility of microbial contamination that can lead to infections; under or over dosing of the drug, either due to errors in measurement, and/or due to loss of drug while crushing the tablets; use of commercially available suspending vehicles can lead to unacceptable interaction between the components of the vehicle and allopurinol, that could also be detrimental to the patient.

However, developing a stable oral suspension of allopurinol presents its own challenges.

Allopurinol is known to have poor aqueous solubility and therefore is prone to sedimentation in suspensions resulting in inconsistent dosing to the patients.

Therefore, there exists a need for developing an oral suspension which addresses the inherent issues of allopurinol, ensures rapid drug release, along with addressing long term stability under varying storage conditions.

The problem is solved by the allopurinol suspension of the present disclosure, which is formulated using the right set of pharmaceutically acceptable excipients, and wherein the allopurinol has a particle size that is capable of providing the required release, so as to provide the desired therapeutic effect, and which is stable over a long period of time, such as 24 months or more. The allopurinol suspension of the present disclosure is also formulated using excipients that provide a viscosity to the suspension that controls caking or sedimentation of the allopurinol, but also allows uniform redispersion to allow uniform dosing of allopurinol to the patient.

Accordingly, the present disclosure provides an oral pharmaceutical suspension comprising:
a. about 10 mg/ml to about 20 mg/ml of allopurinol, and
b. one or more pharmaceutically acceptable excipients,
   wherein 90% of the allopurinol particles in the suspension have a particle size less than 70 microns; the suspension has a viscosity ranging from about 80 mPa·s to about 130 mPa·s; the sedimentation volume is not less than 0.9; and the suspension releases not less than 75% of total allopurinol within15 minutes, when tested using USP Type II apparatus in 900 ml of 0.01N hydrochloric acid at 50 rpm.

The present invention offers a palatable liquid dosage form for patients with swallowing difficulties, as well as for children, thereby improving patient compliance and ease of use. The allopurinol suspension of the present disclosure allows for accurate dose adjustment using a single product, based on therapy goals and outcomes. The suspension has been designed to be bioequivalent to Zyloprim® tablets, and therefore is expected to have no safety and efficacy issues while switching patients from the currently approved tablets to the allopurinol suspension of the present invention.

The allopurinol used in the suspensions of the present disclosure has a defined particle size. In a suspension formulation, the drug is suspended, and therefore, uniformity of dosing is highly dependent on the physical characteristics of the particles. Controlling the particle size of allopurinol in the suspension reduces sedimentation rate, minimizes caking and allows for rapid and easy redispersion with minimal shaking, thereby ensuring consistent dosing with each administration. This is particularly critical in pediatric and geriatric populations where precision in dosing is essential. A defined and controlled particle size that increases the surface area for better dissolution, given the poor solubility of allopurinol in aqueous solutions, is important. Additionally, such a controlled particle size avoids grittiness, making it more palatable, especially for pediatric use.

In one embodiment, the allopurinol has a particle size such that at least 10% of the particles of allopurinol have a size in the range of about 1 micron to about 20 microns, i.e. D10 is in the range of about 1 micron to about 20 microns. In one embodiment, the allopurinol has a particle size such that at least 50% of the particles have a size in the range of about 5 microns to about 30 microns, i.e. D50 is in the range of about 5 microns to about 50 microns. In another embodiment, at least 90% of the particles of allopurinol have a size in the range of about 10 microns to about 50 microns, i.e. D90 is in the range of about 50 microns to about 90 microns. In a highly preferred embodiment, the allopurinol has a particle size of D10 not more than about 20 microns, D50 not more than about 50 microns, and D90 not more than about 70 microns. In yet another embodiment, the allopurinol in the suspension has a particle size such that 10% of the particles have a size less than 20 microns, 50% of the particles have a size less than 50 microns and 90% of the particles have a size less than 70 microns.

The suspension of the present disclosure contains allopurinol at a concentration of about 10 mg/ml to about 20 mg/ml.

The oral allopurinol suspension of the present disclosure has a controlled viscosity limit from about 80 mPa·s to about 130 mPa·s. This range ensures uniform dispersion of the particles by minimizing rapid settling in the suspension, thereby reducing the risk of inconsistent dosage. It also ensures that the consistency of the suspension is suitable for administration to patients, particularly children. The defined viscosity range ensures both, stability under storage conditions, as well as convenience to patients, none of which is addressed by the tablets or conventional compounded suspensions of the prior art.

Settling or sedimentation is a critical issue in suspension stability. The allopurinol suspension of the present disclosure is formulated such that it has controlled caking or sedimentation or settling, and the time taken for redispersion of any sediment is not more than 60 seconds. This ensures that any settled allopurinol sediment can be easily redispersed by simple manual swirling, without the need for any specialised equipment like mechanical centrifuge or rotary shakers, thereby ensuring consistent dosage and minimising effort and time for administration. In one embodiment, the allopurinol suspension is formulated such that the sedimentation volume (the ratio of ultimate volume of sediment to total volume of suspension before settling) is not less than 0.9. A sedimentation volume not less than 0.9 implies that 90% or more of the suspension remains as a uniform dispersion through its shelf life. This is particularly relevant during long term storage of the suspension, when the tendency for particle settling increases.

The allopurinol suspension of the present invention is designed such that not less than 75% of the allopurinol is released within 15 minutes, when tested in USP Type II apparatus using 900 ml of 0.01N hydrochloric acid at 50 rpm. Specifically, for gout management and uric acid reduction, it is imperative to achieve target blood/serum plasma levels for the drug to effectively inhibit xanthine oxidase activity in a timely manner. Such an immediate release of at least 75% of allopurinol from the suspension of the present disclosure under 15 minutes is important to provide the pharmacokinetic parameters necessary for the therapeutic efficacy of allopurinol. As previously mentioned, such an immediate release of the allopurinol from the suspension is also important for it to be bioequivalent to Zyloprim® tablets.

A highly preferred embodiment of the present disclosure provides an oral suspension comprising allopurinol in an amount of about 20 mg/ml and pharmaceutically acceptable excipients, wherein (i) the allopurinol has a particle size such that 90% of the particles are less than 70 microns, (ii) the suspension has a viscosity of about 80 mPaS to about 130 mPaS, (iii) the suspension releases not less than 75% of total allopurinol in 15 minutes, when tested in a USP Type II apparatus using 900 ml of 0.01N hydrochloric acid at 50 rpm, (iv) the sedimentation volume is not less than 0.9 and (v) the pH of the suspension is in the range of about 3.0 to about 6.0.

The allopurinol suspension of the present disclosure includes one or more of solvent, cosolvent, suspending agent, sweetener, flavoring agent, preservative, anti-foaming agent, pH adjusting agent and the like. The suspension may contain one or more excipients from each of the categories listed above.

The solvent used in the suspension of the present disclosure may be selected from water, propylene glycol, glycerin, polyethylene glycol, sorbitol, syrup, polyols such as mannitol, xylitol and mixtures thereof. In most embodiments water is the preferred solvent.

The cosolvent used in the suspension of the present disclosure may be selected from propylene glycol, polyethylene glycol, glycerin, sorbitol, benzyl alcohol, ethanol, polyols, non-anionic surfactants and mixtures thereof. The non-anionic surfactants include, but are not limited to, polysorbates, sorbitan esters, and poloxamers, decyl glucoside, lauryl glucoside, sucrose cocoate caprylyl/capryl glucoside and mixtures thereof. In a preferred embodiment, the cosolvent is propylene glycol. The cosolvent may be used in an amount ranging from about 5% w/w to about 20% w/w.

The one or more suspending agents that may be used in the suspension of the present disclosure may be selected from xanthan gum, carboxymethylcellulose sodium, guar gum, microcrystalline cellulose, bentonite, hydroxypropyl cellulose, hydroxypropyl methyl cellulose, sodium alginate and mixtures thereof, in amount ranging from about 0.1% w/w to about 10% w/w. In preferred embodiments, xanthan gum is used as the suspending agent. Typically, the suspending agent is used in an amount ranging from about 0.1% w/w to about 10% w/w. The amount of suspending agent used is also critical to the viscosity of the suspension and is selected after significant testing. As discussed previously, the present inventors conducted tests to conclude that a viscosity in the range of about 80 mPaS to about 130 mPaS is ideal. The allopurinol suspension of the present disclosure is formulated such that it has controlled caking or sedimentation or settling, and the time taken for redispersion of any sediment is not more than 60 seconds. In one embodiment, the allopurinol suspension is formulated such that the sedimentation volume is not less than 0.9. This is achieved by using pharmaceutically acceptable excipients that impart a viscosity in the range of about 80 mPaS to about 130 mPaS to the allopurinol suspension.

Sweeteners that may be used in the allopurinol suspension of the present invention include one or more agents selected from nutritive sweetening agents, non-nutritive sweetening agents and mixtures thereof. The nutritive sweetening agents may be selected from, but are not limited to, dextrose, fructose, sucrose, agave nectar, brown rice syrup, date sugar, honey, molasses and blackstrap molasses, sorghum syrup, stevia, maple syrup, birch syrup, yacon syrup, lucuma powder, coconut sugar, erythritol, maltitol, mannitol, sorbitol, xylitol, isomalt, lactitol, maltitol and mixtures thereof. The non-nutritive sweetening agents may be selected from, but are not limited to, acesulfame, advantame, alitame, aspartame, neotame, saccharin, sodium saccharin, sucralose, acesulfame potassium, thaumatin, stevioside and mixtures thereof. The sweetener may be used in an amount ranging from about 0.25% w/w to about 10% w/w.

The pH adjusting agents that may be used in the allopurinol suspension of the present disclosure may include buffering agents that may be selected from citric acid, malic acid, succinic acid, fumaric acid, tartaric acid, phosphoric acid, boric acid, ascorbic acid, trisodium citrate, sodium tartarate, sodium acetate, sodium carbonate, sodium polyphosphate, potassium polyphosphate, sodium pyrophosphate, potassium pyrophosphate, potassium citrate, tripotassium citrate, disodium hydrogen phosphate, dipotassium hydrogen phosphate, trisodium phosphate, trisodium citrate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate and the like. In some embodiments, the pH adjusting agent used in the allopurinol suspension described herein comprises a combination of trisodium citrate and citric acid anhydrous. In one embodiment, the pH of the suspension is between about 3.0 to about 6.0.

A flavoring agent or flavorant may be added to enhance the taste or aroma of the allopurinol suspension of the present disclosure. Non-limiting examples of suitable natural flavors, some of which can readily be simulated with synthetic agents or combinations thereof, include almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, mango, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, vanilla, winter green, and the like, as well as combinations thereof. Also useful, particularly where the formulation is intended primarily for pediatric use, is tutti-frutti or bubblegum flavor, a compounded flavoring agent based on fruit flavors. Presently preferred flavoring agents include strawberry, anise, cinnamon, cacao, orange, peppermint, cherry (in particular, wild cherry), grape, bubblegum, vanilla and mixed berry. The flavoring agent may be used in an amount ranging from about 0.01% w/w to about 0.25% w/w.

One or more preservatives that may be used in the allopurinol suspension of the present disclosure may be selected from sodium benzoate, sorbic acid, potassium sorbate, methyl-4-hydroxybenzoate (methyl paraben), propyl-4-hydroxybenzoate (propyl paraben) and mixtures thereof. In preferred embodiments, the preservative used is a combination of methyl paraben and propyl paraben. The preservative may be used in an amount ranging from about 0.005% w/w to about 1% w/w.

The allopurinol suspension may also include an anti-foaming agent, such as simethicone suspension, in an amount ranging from about 0.25% w/w to about 5% w/w.

The allopurinol suspension of the present disclosure is stable for at least 24 months when stored at room temperature. The impurity specification for the suspension is as shown in Table 1 below—

TABLE 1

| Impurity name | Structure | Limit |
|---|---|---|
| Allopurinol related compound A (3-Amino-1Hpyrazole- 4-Carboxamide) | 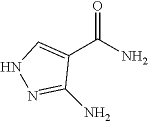 | Not more than 0.2% |
| Allopurinol related compound B (5-(formylamino)-1H- pyrazole-4-carboxamide) | 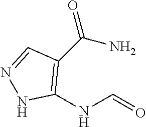 [SMILES: NC(=O)c1cn[nH]c1NC=O] | Not more than 0.2% |
| Allopurinol related compound C (5-(4H-1,2,4-triazol-4-yl)- 1H-pyrazole-4- carboxamide) | 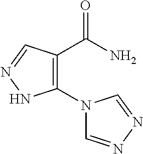 [SMILES: NC(=O)c1cn[nH]c1-n1cnnc1] | Not more than 0.2% |
| Allopurinol related compound D (Ethyl-5-amino-1H- pyrazole- 4-carboxylate) | 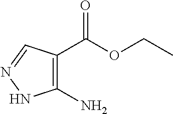 | Not more than 0.2% |
| Allopurinol related compound E (Ethyl-5-(formylamino)- 1H- pyrazole-4-carboxylate) | 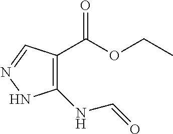 | Not more than 0.2% |

The storage stability studies of the suspension (see examples below) indicate that the product will remain stable for a period of 24 months or more, when stored in amber-colored bottles at room temperature. Such a storage period of 24 months or more is typically referred to as the shelf life of the suspension, i.e. the time during which the suspension will remain stable and can be used without any concerns. In one embodiment of the present disclosure, the oral suspension when stored at a temperature of about 40° C. at relative humidity of 75% and at a temperature of about 250° C. at relative humidity of 60%, the amount of allopurinol in the suspension remains not less than 99.5% of the initial concentration of allopurinol in the suspension, at the end of 3 months. In another embodiment, the oral suspension when stored at a temperature of about 40° C. at relative humidity of 75%, and at a temperature of about 25° C. at relative humidity of 60%, the amount of impurities is not more than 0.05% of the suspension, at the end of 3 months.

The allopurinol suspension of the present disclosure is suitable for titrating the dose of allopurinol that may be needed in a pediatric patient. The suspension is packaged in a suitable container with a dosing syringe with markings that allow measuring the volume accurately. The dosing syringe eases administration of the prescribed dose to the patient, and helps in precise dosing. A printed graduated 20 mL dosing cup may also be included as part of the container closure system to facilitate/support administration of the oral suspension as per the label requirements. The graduations for the dosing cup may have markings at 5, 10, 12.5, 15, 17.5 and 20 mL. These graduations can be used to administer specific dosages of allopurinol suspension of strength 100 mg/5 ml, which can be given in single or divided doses, as shown in

Table 2 below—

TABLE 2

| Total daily dosage (mg) | Divided dose (mg) | Dose volume (ml) |
|---|---|---|
| 100 | — | 5 |
| 200 | — | 10 |
| 300 | — | 15 |
| 400 | 200 | 10 |
| 500 | 250 | 12.5 |
| 600 | 300 | 15 |
| 700 | 350 | 17.5 |
| 800 | 400 | 20 |

An oral dosing dispenser in the form of a graduated syringe or graduated dropper may also be packaged with the product. The allopurinol suspension of the present disclosure has clinical safety and efficacy comparable with Zyloprim®.

### EXAMPLES

### Example 1

An oral allopurinol suspension was prepared as per the details provided Table 3 below—

TABLE 3

| | | Quantity |
|---|---|---|
| Ingredients | Function | (mg/ml) |
| Allopurinol | Active Pharmaceutical | 20 |
| | Ingredient | |
| Propylene glycol | Co-solvent | 100 |
| Sucrose | Sweetener | 25 |
| Xanthan gum | Suspending agent | 3 |
| Propyl paraben | Preservative | 0.1 |
| Methyl paraben | Preservative | 1 |
| Strawberry flavor | Flavoring Agent | 0.5 |
| GUARANA FLAVOR | | |
| 586997 AP0551 | | |
| Sodium saccharin | Sweetener | 5 |
| (15% 10-20 Mesh) | | |
| Simethicone (30% Emulsion) | Antifoaming agent | 1 |
| Trisodium citrate dihydrate | pH adjusters | 3 |
| Citric acid Monohydrate | pH adjusters | 1 |
| Water | Vehicle/Solvent | Qs 1 ml |

Purified water was taken in a jacketed vessel and heated to 65-75° C., and propyl paraben was added to it with continuous stirring, followed by addition of methyl paraben. The solution thus obtained was cooled down to room temperature and Sucrose was added to it while stirring. This was followed by addition of sodium saccharin to get a clear solution, with continuous stirring. Separately, a solution was prepared by taking purified water at room temperature and sequentially adding trisodium citrate dihydrate and citric acid anhydrous to it, with stirring. This citrate solution was added to the previous solution containing sucrose, to obtain an aqueous phase. Propylene glycol was then taken in a high shear mixer, and simethicone emulsion was added to it under continuous high shear mixing, followed by addition of xanthan gum to obtain a suspending phase. The suspending phase was added to the aqueous phase under continuous stirring. The weighed quantity of allopurinol was then added to this at room temperature, with continuous mixing and strawberry flavor was then added to it under continuous homogenization, until a uniform suspension is obtained. Finally, purified water was added to make up the volume, followed by passing the bulk suspension through a cylindrical mesh filter cartridge of 75-micron. The filtered suspension was collected in a holding vessel and stored at 20-30° C. The bulk suspension is filled into 100 mL (4 oz)/24 mm neck and 500 mL (16 oz)/24 mm neck amber colored PET bottles, under continuous mixing. The filled PET bottles were capped with 24 mm SP-410 PP Child Resistance Closure with Induction liner and induction sealed. The bottles were stored at room temperature.

The allopurinol suspension of Example 1 was subjected to stability testing. The results of the same are included in Table 4 below—

TABLE 4

| | Pack (Orientation) | | | | | | | | |
|---|---|---|---|---|---|---|---|---|---|
| | 150 mL/38 mm amber color PET bottles (Upright) | | | | | | | | |
| | Stability Condition | | | | | | | | |
| | | 25 ± 2° C./60 ± 5% RH | | | | 40 ± 2° C./75% RH | | | |
| | | Time Point | | | | | | | |
| | Initial | 2 W | 1 M | 2 M | 3 M | 2 W | 1 M | 2 M | 3 M |
| Test Parameter | Results | | | | | | | | |
| pH | 5.37 | 5.32 | 5.3 | 5.31 | 5.3 | 5.28 | 5.28 | 5.33 | 5.25 |
| Assay of | 102.2 | 100.9 | 101.4 | 98.4 | 97.9 | 101.3 | 101.7 | 100.8 | 98.0 |
| Allopurinol (%) | | | | | | | | | |

Related substances (% w/w)

| | | | | | | | | | |
|---|---|---|---|---|---|---|---|---|---|
| Allopurinol | 0.004 | 0.000 | 0.006 | 0.01 | 0.01 | 0.000 | 0.006 | 0.01 | 0 |
| related | | | | | | | | | |
| compound-A | | | | | | | | | |
| Allopurinol | 0.045 | 0.01 | 0.19 | 0.02 | 0.02 | 0.01 | 0.19 | 0.03 | 0.03 |
| related | | | | | | | | | |
| compound-B | | | | | | | | | |
| Any individual | 0.009 | 0.01 | ND | 0.01 | 0.01 | 0.01 | 0.005 | 0.01 | 0.02 |
| unspecified | | | | | | | | | |
| Impurity | | | | | | | | | |
| Total impurities | 0.07 | 0.02 | 0.2 | 0.04 | 0.04 | 0.02 | 0.2 | 0.05 | 0.05 |
| Specific | 1.0420 | 1.0402 | 1.0389 | 1.0411 | 1.0377 | 1.0402 | 1.0389 | 1.0408 | 1.0409 |
| Gravity | | | | | | | | | |
| Particle size | | | | | | | | | |
| distribution (μ) | | | | | | | | | |
| D10 | 2.9 | 2.4 | 2.75 | 2.96 | 3.401 | 2.10 | 2.73 | 3.29 | 2.46 |
| D50 | 11.1 | 7.6 | 10.02 | 11.33 | 13.24 | 6.20 | 9.3 | 12.48 | 8.14 |
| D90 | 28.1 | 19.5 | 25.33 | 27.55 | 32.58 | 16.25 | 22.17 | 31.61 | 20.71 |
| Sedimentation | 0.99 | 0.99 | 0.99 | 0.99 | 0.99 | 0.99 | 0.99 | 0.99 | 0.99 |
| volume | | | | | | | | | |

### Example 2

An oral suspension of allopurinol as with components as shown in Table 5 below were prepared by a process similar to that disclosed in Example 1 above.

TABLE 5

| | Batch No | | | | | | |
|---|---|---|---|---|---|---|---|
| | 007 | 026 | 029 | 030 | 042 | 043 | 046 |
| Ingredients | mg/mL | mg/mL | mg/mL | mg/mL | mg/mL | mg/mL | mg/mL |
| Allopurinol | 20 | 20 | 20 | 20 | 20 | 20 | 20 |
| (D90 <10 microns) | | | | | | | |
| Propylene Glycol | 100 | 100 | 100 | 100 | 100 | 100 | 100 |
| Sucrose | 50 | 25 | 25 | 25 | 25 | 25 | 25 |
| Xanthan Gum | 3 | 3 | 3 | 3 | 3 | 3 | 3 |
| Propyl Paraben | 0.1 | 0.1 | 0.1 | 0.1 | 0.1 | 0.1 | 0.1 |
| Methyl Paraben | 1 | 1 | 1 | 1 | 1 | 1 | 1 |
| Lemon Flavor | 5 | — | — | — | — | — | — |
| Strawberry | — | 0.5 | 0.5 | 0.5 | 0.5 | 0.5 | 0.5 |
| flavour | | | | | | | |
| Sodium | 5 | 5 | 5 | 5 | 5 | 5 | 5 |
| Saccharin | | | | | | | |
| Simethicone 30 | 1 | 1 | 1 | 1 | 1 | 1 | 1 |
| % Emulsion | | | | | | | |
| FD&C Yellow # 6 | 0.025 | 0.025 | — | — | — | — | — |
| Trisodium citrate | 3 | 3 | 3 | 3 | 3 | 3 | 3 |
| dihydrate | | | | | | | |
| Citric acid | 1 | 1 | 1 | 1 | — | — | — |
| Monohydrate | | | | | | | |
| Citric acid | — | — | — | — | 0.91 | 0.91 | 0.91 |
| Anhydrous | | | | | | | |
| Water | Q.s to | Q.s to | Q.s to | Q.s to | Q.s to | Q.s to | Q.s to |
| | 1 mL | 1 mL | 1 mL | 1 mL | 1 mL | 1 mL | 1 mL |

### Example 3

The allopurinol suspension of strength 100 mg/5 ml was prepared as per the process of Example 1 and was filled in a 100 ml bottle. The suspension was then subjected to stability studies. The results are recorded in Table 6 below.

TABLE 6
Fill volume: 100 mL; Orientation: Invert

| Name of | | | | 25° C. ± 2° C./ | | | | | | | | 40° C. ± 2° C./ | | | | | |
|---|---|---|---|---|---|---|---|---|---|---|---|---|---|---|---|---|---|
| Test | | | | 40 ± 5% RH | | | | | | | | NMT 25% RH | | | | | |
| Parameter | Specification | Initial | | 1 M | | 3 M | | 6 M | | 12 M | | 1 M | | 3 M | | 6 M | |
| Description/ | White to off | Complies | | Complies | | Complies | | Complies | | Complies | | Complies | | Complies | | Complies | |
| Appearance | white suspension | | | | | | | | | | | | | | | | |
| pH | Between 3.0 to | 5.33 | | 5.35 | | 5.23 | | 5.37 | | 5.47 | | 5.35 | | 5.20 | | 5.34 | |
| | 6.0 | | | | | | | | | | | | | | | | |
| Specific | Between 0.979 | 1.0324 | | 1.0297 | | 1.0317 | | 1.0293 | | 1.034 | | 1.0307 | | 1.0313 | | 1.0283 | |
| gravity | to 1.082 | | | | | | | | | | | | | | | | |
| Viscosity | Between 80 to | 107.6 | | 110.73 | | 111.09 | | 110.55 | | 112.34 | | 112.77 | | 110.99 | | 116.61 | |
| | 130 | | | | | | | | | | | | | | | | |
| Particle size | D 10: NMT 20 μm | 5.731 | μm | 5.599 | μm | 5.497 | μm | 5.142 | μm | 5.366 | μm | 5.537 | μm | 5.500 | μm | 5.272 | μm |
| distribution | D 50: NMT 50 μm | 15.080 | μm | 15.888 | μm | 15.709 | μm | 14.961 | μm | 15.768 | μm | 16.303 | μm | 16.148 | μm | 15.774 | μm |
| | D 90: NMT 70 μm | 36.708 | μm | 36.862 | μm | 35.369 | μm | 34.609 | μm | 36.558 | μm | 37.796 | μm | 37.308 | μm | 41.213 | μm |
| Dissolution | Not less than | 108 | | 104 | | 104 | | 104 | | 109 | | 109 | | 104 | | 103 | |
| | 75%(Q) of the | | | | | | | | | | | | | | | | |
| | total allopurinol | | | | | | | | | | | | | | | | |
| | in 15 minutes | | | | | | | | | | | | | | | | |
| Redispers- | NMT 60 | 32 | sec | 30 | sec | 31 | sec | 28 | sec | 31 | sec | 30 | sec | 29 | sec | 30 | sec |

| ibility by | Seconds | | | | | | | | |
|---|---|---|---|---|---|---|---|---|---|
| time | | | | | | | | | |
| Sedimen- | The ratio of | 0.99 | 0.98 | 0.98 | 0.99 | 0.98 | 0.99 | 0.99 | 0.98 |
| tation | ultimate volume | | | | | | | | |
| Volume (F) | of sediment to | | | | | | | | |
| | total volume of | | | | | | | | |
| | suspension | | | | | | | | |
| | before settling | | | | | | | | |
| | should be not | | | | | | | | |
| | less than 0.9 | | | | | | | | |
| Water loss | NMT 5% | NA | 0.16 | 0.55 | 1.05 | 2.07 | 0.54 | 1.88 | 3.56 |
| study | | | | | | | | | |
| Assay of | 80.0 to 110.0% | 102.3 | 102 | 103.7 | 100.8 | 105.6 | 101.7 | 105.1 | 102.6 |
| Methyl | | | | | | | | | |
| Paraben | | | | | | | | | |
| Assay of | 80.0 to 110.0% | 101.2 | 101 | 101.5 | 105.9 | 106.4 | 100.4 | 102.7 | 106.6 |
| Propyl | | | | | | | | | |
| Paraben | | | | | | | | | |
| Assay | Not less than | 104.8 | 103.5 | 104.1 | 104.2 | 104.1 | 104.7 | 104.5 | 103.2 |
| | 90.0% and not | | | | | | | | |
| | more than | | | | | | | | |
| | 110.0% of | | | | | | | | |
| | labelled amount | | | | | | | | |
| | of Allopurinol | | | | | | | | |

Related substances (% w/w)

| | | | | | | | | | |
|---|---|---|---|---|---|---|---|---|---|
| Allopurinol | Not more | ND | 0.01 | 0.02 | 0.02 | 0.02 | 0.02 | 0.03 | 0.06 |
| related | than 0.20% | | | | | | | | |
| Compound- | | | | | | | | | |
| A | | | | | | | | | |
| Allopurinol | Not more | ND | 0.03 | 0.02 | 0.02 | 0.04 | 0.03 | 0.04 | 0.05 |
| related | than 0.20% | | | | | | | | |
| Compound- | | | | | | | | | |
| B | | | | | | | | | |
| Any | Not more | ND | 0.01 | 0.01 | 0.02 | ND | 0.01 | 0.01 | 0.03 |
| individual | than 0.20% | | | | | | | | |
| unspecified | | | | | | | | | |
| degradation | | | | | | | | | |
| product | | | | | | | | | |
| Total | Not more | ND | 0.05 | 0.05 | 0.06 | 0.06 | 0.06 | 0.09 | 0.16 |
| degradation | than 2.0% | | | | | | | | |
| product | | | | | | | | | |

ND: Not Detected; C: Complies; NLT: Not Less Than; NMT: Not More Than. NA: Not Applicable

### Example 4

The allopurinol suspension of strength 100 mg/5 ml was prepared as per the process of Example 1 and was filled in a 450 ml bottle. The suspension was then subjected to stability studies. The results are recorded in Table 7 below.

TABLE 7
Fill volume: 450 mL; Orientation: Invert

| Name of | | | | 25° C. ± 2° C./ | | | | | | | | 40° C. ± 2° C./ | | | | | |
|---|---|---|---|---|---|---|---|---|---|---|---|---|---|---|---|---|---|
| Test | | | | 40 ± 5% RH | | | | | | | | NMT 25% RH | | | | | |
| Parameter | Specification | Initial | | 1 M | | 3 M | | 6 M | | 1 2M | | 1 M | | 3 M | | 6 M | |
| Description/ | White to | Complies | | Complies | | Complies | | Complies | | Complies | | Complies | | Complies | | Complies | |
| Appearance | off white | | | | | | | | | | | | | | | | |
| | suspension | | | | | | | | | | | | | | | | |
| pH | Between | 5.35 | | 5.35 | | 5.24 | | 5.35 | | 5.34 | | 5.34 | | 5.25 | | 5.33 | |
| | 3.0 to 6.0 | | | | | | | | | | | | | | | | |
| Specific | Between | 1.0294 | | 1.0317 | | 1.0300 | | 1.0274 | | 1.007 | | 1.0306 | | 1.0278 | | 1.0215 | |
| gravity | 0.979 to | | | | | | | | | | | | | | | | |
| | 1.082 | | | | | | | | | | | | | | | | |
| Viscosity | Between | 108.12 | | 105.61 | | 105.1 | | 107.49 | | 105.48 | | 106.66 | | 105.89 | | 111.63 | |
| | 80 to 130 | | | | | | | | | | | | | | | | |
| Particle size | D 10: NMT 20 μm | 6.410 | μm | 5.824 | μm | 5.476 | μm | 5.298 | μm | 5.754 | μm | 5.254 | μm | 4.841 | μm | 5.684 | μm |
| distribution | D 50: NMT 50 μm | 16.444 | μm | 16.167 | μm | 15.636 | μm | 14.625 | μm | 16.277 | μm | 15.658 | μm | 14.781 | μm | 15.611 | μm |
| | D 90: NMT 70 μm | 35.680 | μm | 36.488 | μm | 35.580 | μm | 34.240 | μm | 38.496 | μm | 36.425 | μm | 34.604 | μm | 36.020 | μm |
| Dissolution | Not less than | 103 | | 104 | | 101 | | 112 | | 105 | | 106 | | 105 | | 112 | |
| | 75%(Q) of | | | | | | | | | | | | | | | | |
| | the total | | | | | | | | | | | | | | | | |
| | allopurinol | | | | | | | | | | | | | | | | |
| | in 15 | | | | | | | | | | | | | | | | |
| | minutes | | | | | | | | | | | | | | | | |
| Redispers- | NMT 60 | 32 | sec | 31 | sec | 30 | sec | 28 | sec | 39 | sec | 30 | sec | 30 | sec | 29 | sec |

| ibility by | Seconds | | | | | | | | |
|---|---|---|---|---|---|---|---|---|---|
| time | | | | | | | | | |
| Sedimen- | The ratio | 0.99 | 0.99 | 0.99 | 0.99 | 0.99 | 0.99 | 0.98 | 0.99 |
| tation | of ultimate | | | | | | | | |
| Volume (F) | volume of | | | | | | | | |
| | sediment to | | | | | | | | |
| | total | | | | | | | | |
| | volume of | | | | | | | | |
| | suspension | | | | | | | | |
| | before | | | | | | | | |
| | settling | | | | | | | | |
| | should be | | | | | | | | |
| | not less | | | | | | | | |
| | than 0.9 | | | | | | | | |
| Water loss | NMT 5% | NA | 0.28 | 0.45 | 0.70 | 1.28 | NP | NP | NP |
| study | | | | | | | | | |
| Assay of | 80.0 to | 101.5 | 99.9 | 103.6 | 101.8 | 102.8 | 100.6 | 102.3 | 102.9 |
| Methyl | 110.0% | | | | | | | | |
| Paraben | | | | | | | | | |
| Assay of | 80.0 to | 100.3 | 99.4 | 101.5 | 104.3 | 105.7 | 100.1 | 100.4 | 104.7 |
| Propyl | 110.0% | | | | | | | | |
| Paraben | | | | | | | | | |
| Assay of | NLT 90.0% | 102.7 | 104.1 | 104.2 | 103.8 | 103.5 | 104.2 | 104.6 | 104.6 |
| Allopurinol | and NMT | | | | | | | | |
| | 110.0% of | | | | | | | | |
| | labelled | | | | | | | | |
| | amount of | | | | | | | | |
| | Allopurinol | | | | | | | | |

Related substances (% w/w)

| | | | | | | | | | |
|---|---|---|---|---|---|---|---|---|---|
| Allopurinol | Not more | 0.01 | 0.01 | 0.01 | 0.02 | 0.02 | 0.01 | 0.03 | 0.06 |
| related | than 0.2% | | | | | | | | |
| Compound- | | | | | | | | | |
| A | | | | | | | | | |
| Allopurinol | Not more | 0.02 | 0.02 | 0.02 | 0.03 | 0.02 | 0.03 | 0.04 | 0.05 |
| related | than 0.2% | | | | | | | | |
| Compound- | | | | | | | | | |
| B | | | | | | | | | |
| Any | Not more | ND | 0.01 | 0.01 | 0.02 | ND | 0.01 | 0.01 | 0.03 |
| individual | than 0.2% | | | | | | | | |
| unspecified | | | | | | | | | |
| degradation | | | | | | | | | |
| product | | | | | | | | | |
| Total | Not more | 0.03 | 0.04 | 0.04 | 0.07 | 0.04 | 0.05 | 0.09 | 0.15 |
| degradation | than 2.0% | | | | | | | | |
| product | | | | | | | | | |

ND: Not Detected; NLT: Not Less Than; NMT: Not More Than, NP: Not Performed, NA: Not Applicable.

### Example 5

The allopurinol suspension of strength 100 mg/5 ml was prepared as per the process of Example 2 (batches prepared with Citric acid anhydrous 0.91 mg/mL) and was filled in a 100 ml bottle. The suspension was then subjected to stability studies. The results are recorded in Table 8 below.

TABLE 8
Fill volume: 100 mL, Orientation: Invert

| Name of | | | | 25° C. ± 2° C./ | | | | | | | | 40° C. ± 2° C./ | | | | | |
|---|---|---|---|---|---|---|---|---|---|---|---|---|---|---|---|---|---|
| Test | | | | 40 ± 5% RH | | | | | | | | NMT 25% RH | | | | | |
| Parameter | Specification | Initial | | 1 M | | 3 M | | 6 M | | 9 M | | 1 M | | 3 M | | 6 M | |
| Description/ | White to off | WCS | | WCS | | WCS | | WCS | | WCS | | WCS | | WCS | | WCS | |
| Appearance | white | | | | | | | | | | | | | | | | |
| | suspension | | | | | | | | | | | | | | | | |
| pH | Between 3.0 to | 5.27 | | 5.37 | | 5.40 | | 5.36 | | 5.32 | | 5.36 | | 5.38 | | 5.35 | |
| | 6.0 | | | | | | | | | | | | | | | | |
| Specific | Between 0.979 | 1.0290 | | 1.0312 | | 1.0319 | | 1.024 | | 1.016 | | 1.0302 | | 1.0315 | | 1.026 | |
| gravity | to 1.082 | | | | | | | | | | | | | | | | |
| Viscosity | Between 80 to | 106.47 | | 103.01 | | 105.65 | | 107.64 | | 105.8 | | 104.43 | | 107.25 | | 107.21 | |
| | 130 | | | | | | | | | | | | | | | | |
| Particle size | D 10: NMT 20 μm | 5.697 | μm | 5.619 | μm | 5.292 | μm | 5.604 | μm | 5.516 | μm | 5.528 | μm | 5.186 | μm | 5.603 | μm |
| distribution | D 50: NMT 50 μm | 16.143 | μm | 16.334 | μm | 15.167 | μm | 16.119 | μm | 16.399 | μm | 16.482 | μm | 15.548 | μm | 16.475 | μm |
| | D 90: NMT 70 μm | 38.041 | μm | 37.881 | μm | 35.180 | μm | 37.702 | μm | 41.266 | μm | 38.543 | μm | 36.744 | μm | 38.475 | μm |
| Dissolution | Not less than | 105 | | 100 | | 107 | | 107 | | 101 | | 101 | | 110 | | 111 | |
| | 75%(Q) of the | | | | | | | | | | | | | | | | |
| | total allopurinol | | | | | | | | | | | | | | | | |
| | in 15 minutes | | | | | | | | | | | | | | | | |
| Redispers- | NMT 60 Sec | 32 | sec | 30 | sec | 28 | sec | 29 | sec | 29 | sec | 31 | sec | 29 | sec | 31 | sec |

| ibility by | | | | | | | | | |
|---|---|---|---|---|---|---|---|---|---|
| time | | | | | | | | | |
| Sedimen- | The ratio of | 0.98 | 0.98 | 0.98 | 0.99 | 0.99 | 0.98 | 0.98 | 0.99 |
| tation | ultimate | | | | | | | | |
| Volume (F) | volume of | | | | | | | | |
| | sediment to | | | | | | | | |
| | total volume of | | | | | | | | |
| | suspension | | | | | | | | |
| | before settling | | | | | | | | |
| | should be not | | | | | | | | |
| | less than 0.9 | | | | | | | | |
| Water loss | NMT 5% | NA | 0.18 | 0.51 | 0.99 | 1.48 | 0.49 | 1.51 | 3.03 |
| study | | | | | | | | | |
| Assay of | 80.0 to 110.0% | 103.1 | 102.7 | 103.1 | 104.4 | 103.5 | 101.2 | 104.7 | 105.8 |
| Methyl | | | | | | | | | |
| Paraben | | | | | | | | | |
| Assay of | 80.0 to 110.0% | 100.0 | 98.2 | 102.1 | 102.9 | 98.9 | 99.1 | 102.8 | 105.1 |
| Propyl | | | | | | | | | |
| Paraben | | | | | | | | | |
| Assay of | Not less than | 103.7 | 101.3 | 103.3 | 103.4 | 104.5 | 101.2 | 104.1 | 104.5 |
| Allopurinol | 90.0% and not | | | | | | | | |
| | more than | | | | | | | | |
| | 110.0% of | | | | | | | | |
| | labelled amount | | | | | | | | |
| | of Allopurinol | | | | | | | | |

Related Substances (% w/w)

| Allopurinol | Not more than | 0.01 | 0.01 | 0.01 | 0.01 | 0.02 | 0.02 | 0.03 | 0.04 |
|---|---|---|---|---|---|---|---|---|---|
| related | 0.20% | | | | | | | | |
| Compound- | | | | | | | | | |
| A | | | | | | | | | |
| Allopurinol | Not more than | 0.02 | 0.02 | 0.02 | ND | 0.02 | 0.03 | 0.04 | ND |
| related | 0.20% | | | | | | | | |
| Compound- | | | | | | | | | |
| B | | | | | | | | | |
| Any | Not more than | 0.01 | 0.01 | 0.01 | ND | ND | 0.01 | 0.01 | ND |
| individual | 0.20% | | | | | | | | |
| unspecified | | | | | | | | | |
| degradation | | | | | | | | | |
| product | | | | | | | | | |
| Total | Not more than | 0.04 | 0.04 | 0.04 | 0.01 | 0.04 | 0.06 | 0.09 | 0.04 |
| degradation | 2.0% | | | | | | | | |
| product | | | | | | | | | |

ND: Not Detected; WCS: White color suspension; NLT: Not Less Than; NMT: Not More Than, NA; Not Applicable, M: Month.

### Example 6

The allopurinol suspension of strength 100 mg/5 ml was prepared as per the process of Example 2 (batches prepared with Citric acid anhydrous 0.91 mg/mL) and was filled in a 450 mL bottle. The suspension was then subjected to stability studies. The results are recorded in Table 9 below.

TABLE 9
Fill volume: 450 mL Orientation: Invert

| Name of | | | | 25° C. ± 2° C./ | | | | | | | | 40° C. ± 2° C./ | | | | | |
|---|---|---|---|---|---|---|---|---|---|---|---|---|---|---|---|---|---|
| Test | | | | 40 ± 5% RH | | | | | | | | NMT 25% RH | | | | | |
| Parameter | Specification | Initial | | 1 M | | 3 M | | 6 M | | 9 M | | 1 M | | 3 M | | 6 M | |
| Description/ | White to off white | WCS | | WCS | | WCS | | WCS | | WCS | | WCS | | WCS | | WCS | |
| Appearance | suspension | | | | | | | | | | | | | | | | |
| pH | Between 3.0 to 6.0 | 5.40 | | 5.37 | | 5.40 | | 5.37 | | 5.32 | | 5.37 | | 5.39 | | 5.35 | |
| Specific | Between 0.979 to | 1.0287 | | 1.0294 | | 1.0319 | | 1.023 | | 1.008 | | 1.0306 | | 1.0305 | | 1.016 | |
| gravity | 1.082 | | | | | | | | | | | | | | | | |
| Viscosity | Between 80 to 130 | 103.92 | | 100.78 | | 100.77 | | 104.85 | | 100.86 | | 100.38 | | 101.40 | | 100.49 | |
| Particle | D 10: NMT 20 μm | Mean (μ) | | “Mean(μ) | | Mean(μ) | | Mean(μ) | | Mean(μ) | | Mean(μ) | | Mean(μ) | | Mean(μ) | |
| size | D 50: NMT 50 μm | 5.743 | μm | 5.735 | μm | 5.550 | μm | 5.437 | μm | 5.419 | μm | 6.019 | μm | 5.311 | μm | 5.478 | μm |
| distribution | D 90: NMT 70 μm | 16.224 | μm | 16.229 | μm | 15.403 | μm | 15.788 | μm | 16.212 | μm | 16.385 | μm | 15.099 | μm | 15.497 | μm |
| | | 36.427 | μm | 37.357 | μm | 34.733 | μm | 36.204 | μm | 42.395 | μm | 34.755 | μm | 34.603 | μm | 35.196 | μm |
| Dissolution | Not less than | 105 | | 98 | | 103 | | 106 | | 99 | | 100 | | 110 | | 106 | |
| | 75%(Q) of the total | | | | | | | | | | | | | | | | |
| | allopurinol | | | | | | | | | | | | | | | | |
| | in 15 minutes | | | | | | | | | | | | | | | | |
| Redispers- | NMT 60 Sec | 32 | sec | 32 | sec | 30 | sec | 30 | sec | 30 | sec | 30 | sec | 31 | sec | 30 | sec |

| ibility by | | | | | | | | | |
|---|---|---|---|---|---|---|---|---|---|
| time | | | | | | | | | |
| Sedimen- | The ratio of ultimate | 0.98 | 0.98 | 0.99 | 0.98 | 0.98 | 0.99 | 0.99 | 0.99 |
| tation | volume of sediment | | | | | | | | |
| Volume (F) | to total volume of | | | | | | | | |
| | suspension before | | | | | | | | |
| | settling should be | | | | | | | | |
| | not less than 0.9 | | | | | | | | |
| Water loss | NMT 5% | NA | 0.09 | 0.24 | 0.49 | 0.74 | 0.26 | 0.76 | 1.51 |
| study | | | | | | | | | |
| Assay of | 80.0 to 110.0% | 100.9 | 99.0 | 99.8 | 100.9 | 100.5 | 98.8 | 98.1 | 100.7 |
| Methyl | | | | | | | | | |
| Paraben | | | | | | | | | |
| Assay of | 80.0 to 110.0% | 100.8 | 99.1 | 101.6 | 102.9 | 99.8 | 99.3 | 103.3 | 103.3 |
| Propyl | | | | | | | | | |
| Paraben | | | | | | | | | |
| Assay of | NLT 90.0% and | 104.4 | 100.9 | 99.8 | 102.6 | 101.6 | 100.9 | 102.8 | 104.5 |
| Allopurinol | NMT 110.0% of | | | | | | | | |
| | labelled amount | | | | | | | | |
| | of Allopurinol | | | | | | | | |

Related Substances (% w/w)

| Allopurinol | Not more | 0.01 | 0.01 | 0.01 | 0.02 | 0.02 | 0.02 | 0.03 | 0.05 |
|---|---|---|---|---|---|---|---|---|---|
| related | than 0.20% | | | | | | | | |
| Compound- | | | | | | | | | |
| A | | | | | | | | | |
| Allopurinol | Not more | 0.02 | 0.02 | 0.02 | ND | 0.02 | 0.03 | 0.04 | ND |
| related | than 0.20% | | | | | | | | |
| Compound- | | | | | | | | | |
| B | | | | | | | | | |
| Any | Not more | 0.01 | 0.01 | 0.01 | ND | ND | 0.01 | 0.02 | ND |
| individual | than 0.20% | | | | | | | | |
| unspecified | | | | | | | | | |
| degradation | | | | | | | | | |
| product | | | | | | | | | |
| Total | Not more | 0.04 | 0.04 | 0.04 | 0.02 | 0.04 | 0.06 | 0.09 | 0.05 |
| degradation | than 2.0% | | | | | | | | |
| product | | | | | | | | | |

M: Month,
NMT: Not More Than,
ND: Not Detected,
NA: Not Applicable,
WCS: White Color Suspension

We claim:

1. An oral pharmaceutical suspension comprising:
a. about 10 mg/ml to about 20 mg/ml of allopurinol, and
b. one or more pharmaceutically acceptable excipients,
wherein 90% of the allopurinol particles in the suspension have a particle size less than about 70 microns; the suspension has a viscosity ranging from about 80mPaS to about 130 mPaS; the sedimentation volume is not less than 0.9; and the suspension releases not less than 75% of total allopurinol within 15 minutes, when tested using USP Type II apparatus in 900 ml of 0.01N hydrochloric acid at 50 rpm.

2. The oral suspension as claimed in claim 1, wherein the one or more pharmaceutically acceptable excipients are selected from a group comprising solvents, cosolvents, suspending agents, sweeteners, pH adjusting agents, flavoring agents, preservatives and anti-foaming agents.

3. The oral suspension as claimed in claim 2, wherein:
a. the solvent is selected from a group comprising water, propylene glycol, glycerin, polyethylene glycol, sorbitol, syrup, polyols and mixtures thereof;
b. the cosolvent is selected from a group comprising propylene glycol, polyethylene glycol, glycerin, sorbitol, benzyl alcohol, ethanol, polyols, non-anionic surfactants and mixtures thereof;
c. the suspending agent is selected from a group comprising xanthan gum, carboxymethylcellulose sodium, guar gum, microcrystalline cellulose, bentonite, hydroxypropyl cellulose, hydroxypropyl methyl cellulose, sodium alginate and mixtures thereof;
d. the sweetener is selected from a group comprising nutritive sweeteners including dextrose, fructose, sucrose, agave nectar, brown rice syrup, date sugar, honey, molasses blackstrap molasses, sorghum syrup, stevia, maple syrup, birch syrup, yacon syrup, lucuma powder, coconut sugar, erythritol, maltitol, mannitol, sorbitol, xylitol, isomalt, lactitol, maltitol and mixtures thereof and non-nutritive sweeteners including acesulfame, advantame, alitame, aspartame, neotame, saccharin, sodium saccharin, sucralose, acesulfame potassium, thaumatin, stevioside and mixtures thereof;
e. the pH adjusting agent is selected from a group comprising citric acid, malic acid, succinic acid, fumaric acid, tartaric acid, phosphoric acid, boric acid and ascorbic acid, trisodium citrate, sodium tartarate, sodium acetate, sodium carbonate, sodium polyphosphate, potassium polyphosphate, sodium pyrophosphate, potassium pyrophosphate, potassium citrate, tripotassium citrate, disodium hydrogen phosphate, dipotassium hydrogen phosphate, trisodium phosphate, trisodium citrate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts and pharmaceutically acceptable salts thereof;
f. the flavoring agent is selected from a group comprising almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, mango, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, Vanilla, winter green, bubble gum flavour and combinations thereof;
g. the preservative is selected from a group comprising sodium benzoate, sorbic acid/potassium sorbate, methyl-4-hydroxybenzoate (methyl paraben), propyl-4-hydroxybenzoate (propyl paraben) and mixtures thereof; and
h. the anti-foaming agent is a simethicone suspension.

4. The oral suspension as claimed in claims 2 and 3, wherein the solvent is present in an amount ranging from about 0.5% w/w to about 95% w/w.

5. The oral suspension as claimed in claims 2 and 3, wherein the suspending agent is present in an amount ranging from about 0.1% w/w to about 10% w/w.

6. The oral suspension as claimed in claims 2 and 3, wherein the anti-foaming agent is present in an amount ranging from about 0.25% w/w to about 5% w/w.

7. The oral suspension as claimed in claims 2 and 3, wherein the co-solvent is present in an amount ranging from about 5% w/w to about 20% w/w.

8. The oral suspension as claimed in claims 2 and 3, wherein the sweetener is present in an amount ranging from about 0.25% w/w to about 10% w/w.

9. The oral suspension as claimed in claims 2 and 3, wherein the flavoring agent is present in an amount ranging from about 0.01% w/w to about 0.25% w/w.

10. The oral suspension as claimed in claims 2 and 3, wherein the preservative is present in an amount ranging from about 0.005% w/w to about 1.0% w/w.

11. The oral suspension as claimed in claim 1, wherein the pH of the suspension is in the range of about 3 to about 6.

12. The oral suspension as claimed in claim 1, wherein the suspension remains stable for at least 24 months, when stored at room temperature.

13. The oral suspension as claimed in claim 1, wherein upon storage at a temperature of about 40° C. and relative humidity of about 75%, and at a temperature of about 25° C. and relative humidity of about 60%, the amount of allopurinol in the suspension remains not less than 99.5% of the initial concentration of allopurinol in the suspension, at the end of 3 months.

14. The oral suspension as claimed in claim 1, wherein upon storage at a temperature of about 40° C. and relative humidity of 75% and at a temperature of about 25° C. and relative humidity of 60%, the amount of impurities is not more than 0.05% of the suspension, at the end of 3 months.

15. The oral suspension as claimed in claim 1, wherein the allopurinol has a particle size such that 10% of the particles have a size less than 20 microns, 50% of the particles have a size less than 50 microns and 90% of the particles have a size less than 70 microns.
